(19) United States
(12) Patent Application Publication
(10) Pub. No.: US 2011/0226621 A1
(43) Pub. Date: Sep. 22, 2011

(54) TARGET RECOGNITION MOLECULE AND A METHOD FOR IMMOBILIZING THE SAME

(75) Inventor: Yuichiro Shimizu, Osaka (JP)

(73) Assignee: Sharp Kabushiki Kaisha, Osaka (JP)

(21) Appl. No.: 12/976,805

(22) Filed: Dec. 22, 2010

(30) Foreign Application Priority Data
Dec. 25, 2009 (JP) 2009-296106

(51) Int. Cl. G01N 33/68; C07K 2/00; C08F 8/32; C07K 5/08; C07K 7/06; C07K 5/10; C07K 7/08; G01N 27/447

(52) U.S. Cl. 204/451; 530/300; 525/54.1; 530/322; 530/331; 530/329; 530/330; 530/328; 530/327; 530/326; 204/600

(57) ABSTRACT

There is provided a novel target recognition molecule. The target recognition molecule has a specific reactivity, and can be densely self-assembled and immobilized reversibly or irreversibly at a predetermined site in a microfluidic device. And The target recognition molecule including: (1) a target recognition peptide segment having an amino acid sequence which specifically interacts with a target substance capable of causing an immune reaction; and (2) an electrostatically-charged segment which is provided with three or more electrostatically-charged functional groups capable of being electrically charged to the same polarity in the same solution.

### BACKGROUND OF THE INVENTION

1. Field of the Invention

This invention generally relates to a target recognition molecule having a binding site which specifically interacts with a target substance capable of causing an immune reaction, and it more specifically relates to a target recognition molecule which is imparted with an electrostatic property so that it is densely self-assembled and immobilized at a specific site in a microfluidic device.

2. Background Art

In recent years, an analytical microfluidic device using a chip, in which target recognition molecules selectively and specifically interacting with a target substance capable of causing an immune reaction are immobilized in a microchannel, has been widely used for the microanalysis of a substance such as protein and DNA in a biologic sample.

As such a target recognition molecule, naturally-derived antibodies have been used in the past. Recently, artificial antibodies formed of synthetic peptides or other like compounds have been used from the aspect of their long-term storability, productivity and so on.

Such a type of microfluidic device, in which target recognition molecules selectively and specifically reacting with a target substance capable of causing an immune reaction are immobilized, provides an advantage of being easy to manipulate and requiring no high level of analytical skill, and an advantage capable of assaying a target substance in a short period of time with a less test substance volume. On the other hand, for example, it is not easy to properly immobilize and hold a required amount of target recognition molecule at a predetermined spot, therefore there is not always obtained an assay accuracy, reliability, or reproducibility of satisfactory level.

In regard to the method for immobilization of a target recognition molecule, a variety of methods have been proposed heretofore. For example, one known method is to cause a target recognition molecule to be physically adsorbed on the surface of a base material, and another known method is to cause a target recognition molecule to be covalently linked to the surface of a base material. In addition, there is still another known method in which a target recognition molecule is immobilized on the surface of a minute bead and the minute bead is placed in a microchannel. Furthermore, there are other known methods as set forth in the following conventional art literatures.

### CITATION LIST

### Patent Literatures

Patent Literature 1: JP-A-2003-344396
Patent Literature 2: JP-A-2006-266831
Patent Literature 3: JP-T-04-501605
Patent Literature 4: JP-A-2000-266716
Patent Literature 5: JP-A-2006-71324
Patent Literature 6: JP-A-04-331362

### SUMMARY OF THE INVENTION

For the case of a microfluidic device in which target recognition molecules selectively and specifically interacting with a target substance are immobilized in a certain site, the productivity of the microfluidic device depends much on the efficiency of immobilization and, in addition, its analytical performance depends much on whether the immobilization density or the immobilization state of target recognition molecules is good or bad. If the immobilization efficiency of target recognition molecules is low, lots of time is required for the immobilization since most of the target recognition molecules are not immobilized and are removed.

In addition, the low immobilization efficiency does not provide desired immobilization density, and thus sufficient analytical sensitivity is not obtained. Hence, there have been demands for means by which target recognition molecules can be rapidly and adequately immobilized with high density at a predetermined site in a microchannel.

Furthermore, some target recognition molecules are inferior in chemical/physical stability. When a microfluidic device is used in which such target recognition molecules inferior in chemical/physical stability are immobilized, there is caused a problem of its short life. One of methods for overcoming this problem is a method in which target recognition molecules are in situ immobilized at the time of analysis. To this end, however, there is required a means capable of simply immobilizing target recognition molecules on the spot of analysis.

Even using the methods described in the Patent Literatures 1 to 5, peptide molecules could be also immobilized. However, it is difficult for these methods to immobilize peptide molecules with a low molecular weight in such a density that a desired signal level is obtained.

The method according to the Patent Literature 6 can hardly provide a sufficient speed or amount of electrodeposition. Especially in the case of a peptide having a low molecular weight, it is difficult to densely electrodeposit and immobilize it with high reproducibility. The reason is that an artificially produced peptide with a low molecular weight has less immobilization sites compared with a natural antibody.

The present invention has been made in order to solve the above problems.

A first object of the present invention is to provide a novel target recognition molecule having both functions allowing for target recognition and highly dense immobilization.

A second object of the present invention is to provide a technology for efficiently immobilizing a target recognition molecule onto a base material.

A third object of the present invention is to provide an electrode plate on which the novel target recognition molecule of the present invention is immobilized, and to provide a specific molecule detection apparatus using this electrode plate.

The present invention is directed to a novel target recognition molecule into which an electrostatically-charged segment has been incorporated, and a group of aspects of the present invention are configured as described below.

### (1) Target Recognition Molecule According to a First Aspect of the Present Invention

A target recognition molecule comprising: a target recognition peptide segment having an amino acid sequence which specifically interacts with a target substance capable of causing an immune reaction; and an electrostatically-charged segment which does not specifically interact with the target substance and which is provided with three or more electrostatically-charged functional groups capable of being electrically charged with charges of the same polarity in the same solution.

The electrostatically-charged segment in the target recognition molecule charges more strongly in a solution than other segments. The above “electrostatically-charged functional groups capable of being electrically charged with charges of the same polarity in the same solution” means a functional group that charges positively or negatively within the target recognition molecule when the molecule is immersed in a solution and then dissociated. The above three or more electrostatically-charged functional groups may be composed of only identical functional groups or different functional groups from each other.

The target recognition peptide segment means a peptide which is specifically linked with a target substance capable of causing an immune reaction. Whether a peptide has the linkability or not can be determined using a conventional immune method. For example, the use of quantitative linkage assays such as ELISA method, western blot method, SPR and QCM allows to determine the linkability.

The target recognition peptide segment may be produced by organisms using gene recombination, or may be synthesized chemically. The method used may be selected from known techniques. In the case of gene recombination, after determining a DNA base sequence that generates a RNA codon corresponding to the intended amino acid sequence, the DNA base sequence is synthesized using a known DNA synthesis technique. The resulting DNA base sequence is introduced into a virus vector and thereby infected to a target cell. Thus, the peptide of the target recognition peptide segment is produced using a biological method.

Meanwhile, as the chemical synthesis method, either solid-phase peptide synthesis or liquid-phase peptide synthesis is adopted. Using an amino acid in which functional groups of its side chain and its α-amino group are protected with protective groups so as to allow a desired linkage, an amino acid chain is synthesized and extended to a desired sequence. Thereafter, the protective groups are released and thus the intended target recognition peptide segment is obtained. The reaction for protection or deprotection of protective groups may use a known method.

### (2) Second Aspect

In addition, in accordance with a second aspect of the present invention, there is provided a target recognition molecule according to the aforesaid first aspect wherein an electrostatically-charged segment which is not provided with any functional groups capable of being electrically charged with charges of a different polarity in the same solution.

In the above configuration, it is easy to electrically control movement of the target recognition molecule through the electrostatically-charged segment because all electrostatically-charged functional groups in the electrostatically-charged segment are charged with the same polarity.

### (3) Third Aspect

In addition, in accordance with a third aspect of the present invention, there is provided a target recognition molecule according to either the aforesaid first or second aspect wherein the electrostatically-charged segment is directly linked to an amino acid constituting the target recognition peptide segment.

### (4) Fourth Aspect

Still in addition, in accordance with a fourth aspect of the present invention, there is provided a target recognition molecule according to any one of the aforesaid first, second and third aspects wherein an average isoelectric point of the target recognition peptide segment is 6 or less, and the three or more electrostatically-charged functional groups of the electrostatically-charged segment can charge negatively in a solution of pH 6.5 or more.

### (5) Fifth Aspect

Still in addition, in accordance with a fifth aspect of the present invention, there is provided a target recognition molecule according to any one of the aforesaid first, second and third aspects wherein an average isoelectric point of the target recognition peptide segment is 8 or more, and the three or more electrostatically-charged functional groups of the electrostatically-charged segment can charge positively in a solution of pH 7.5 or less.

### (6) Sixth Aspect

Still in addition, in accordance with a sixth aspect of the present invention, there is provided a target recognition molecule according to any one of the aforesaid first, second and third aspects wherein an average isoelectric point of the target recognition peptide segment is more than 6 and less than 8, and the three or more electrostatically-charged functional groups of the electrostatically-charged segment can charge negatively in a solution of pH 6.5 or more, or positively in a solution of pH 7.5 or less.

Here, what is meant by “an average isoelectric point” is an average value of isoelectric points which is defined by a value (average value) found as a result of division of a value which is a combined value of the isoelectric points of amino acids corresponding to individual amino acid residues forming a target recognition peptide segment (sum value) by the number of the amino acid residues. The isoelectric point of each of the amino acids is as shown in Table 1.

TABLE 1

| amino acid class | abbrev. form | isoelectric point |
|---|---|---|
| alanine | A (Ala) | 6.00 |
| arginine | R (Arg) | 10.76 |
| asparagine | N (Asx) | 5.41 |
| aspartic acid | D (Asp) | 2.77 |
| cysteine | C (Cys) | 5.05 |
| glutamine | Q (Glx) | 5.65 |
| glutamic acid | E (Glu) | 3.22 |
| glycine | G (Gly) | 5.97 |
| histidine | H (His) | 7.59 |
| isoleucine | I (Ile) | 6.05 |
| leucine | L (Leu) | 5.98 |
| lysine | K (Lys) | 9.75 |
| methionine | M (Met) | 5.74 |
| phenylalanine | F (Phe) | 5.48 |
| proline | P (Pro) | 6.30 |
| serine | S (Ser) | 5.68 |
| threonine | T (Thr) | 6.16 |
| tryptophan | W (Trp) | 5.89 |
| tyrosine | Y (Tyr) | 5.66 |
| valine | V (Val) | 5.96 |

### [Non-Peptide Electrostatically-Charged Segment]

### (7) Seventh Aspect

In accordance with a seventh aspect of the present invention, there is provided a target recognition molecule according to any one of the aforesaid first, second, third, fourth or sixth aspects wherein the electrostatically-charged segment is a segment which has a polyacrylic acid building block represented by the following chemical formula (1) with n being not less than 3 nor more than 150.

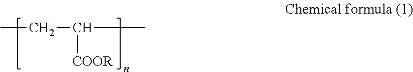

wherein R is H, Na, or K.

### (8) Eighth Aspect

Still in addition, in accordance with an eighth aspect of the present invention, there is provided a target recognition molecule according to any one of the aforesaid first, second, third, fourth or sixth aspects wherein the electrostatically-charged segment is a segment having a polystyrene sulfonic acid building block represented by the following chemical formula (2) with n being not less than 3 nor more than 150.

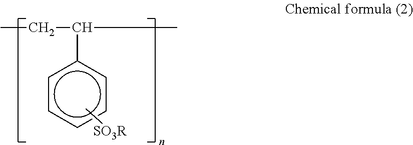

wherein R is H, Na, or K.

### (9) Ninth Aspect

Still in addition, in accordance with a ninth aspect of the present invention, there is provided a target recognition molecule according to any one of the aforesaid first, second, third, fourth and sixth aspects wherein the electrostatically-charged segment is a segment having a polyvinyl sulfate building block represented by the following chemical formula (3) with n being not less than 3 nor more than 150.

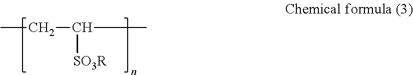

wherein R is H, Na, or K.

### (10) Tenth Aspect

Still in addition, in accordance with a tenth aspect of the present invention, there is provided a target recognition molecule according to any one of the aforesaid first, second, third, fourth and sixth aspects wherein the electrostatically-charged segment is a segment having a polydextran sulfate building block represented by the following chemical formula (4) with n being not less than 1 nor more than 150.

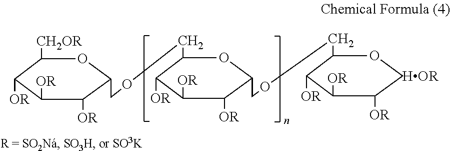

### (11) Eleventh Aspect

Still in addition, in accordance with an eleventh aspect of the present invention, there is provided a target recognition molecule according to any one of the aforesaid first, second, third, fourth and sixth aspects wherein the electrostatically-charged segment is a segment having a polychondroitin sulfate building block represented by the following chemical formula (5) with n being not less than 1 nor more than 150.

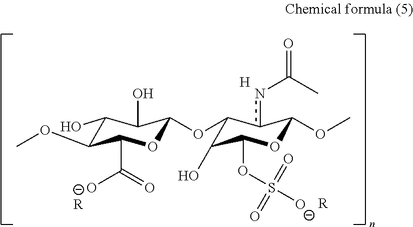

wherein R is H, Na, or K.

### (12) Twelfth Aspect

Still in addition, in accordance with a twelfth aspect of the present invention, there is provided a target recognition molecule according to any one of the aforesaid first, second, third, fourth and sixth aspects wherein the electrostatically-charged segment is a segment having a polynucleotide building block represented by the following chemical formula (6) with n being not less than 3 nor more than 150.

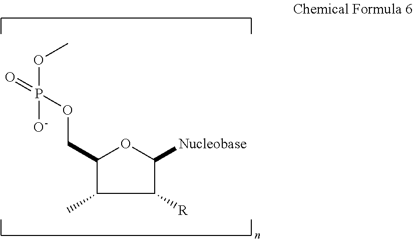

wherein R is H or OH.

Since a nucleotide, composed of a phosphoric acid, a sugar (either ribose (R═OH) or deoxyribose (R═H)), and bases (adenine, cytosine, guanine, thymine (only for deoxyribose), uracil (only for ribose)), has a phosphoric acid content, it becomes electrically charged with negative charges in a basic solution. Therefore, the target recognition molecule of this configuration is suitable for the analysis of a target substance that employs a solution of from alkali to mild acidic. In addition, a single stranded polynucleotide (such as ssDNA and ssRNA) may be used as an electrostatically-charged segment. Alternatively, a double stranded polynucleotide (dsDNA) may be used as an electrostatically-charged segment.

### (13) Thirteenth Aspect

Still in addition, in accordance with a thirteenth aspect of the present invention, there is provided a target recognition molecule according to any one of the aforesaid first, second, third, fifth and sixth aspects wherein the electrostatically-charged segment is a segment having a polyethylenimine building block represented by the following chemical formula (7).

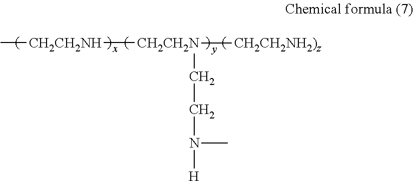

wherein x: y: z=0.5: 0.25: 0.25 and [x+y+z] is an integer not less than 3 nor more than 150.

### (14) Fourteenth Aspect

Still in addition, in accordance with a fourteenth aspect of the present invention, there is provided a target recognition molecule according to any one of the aforesaid first, second, third, fifth and sixth aspects wherein the electrostatically-charged segment is a segment having a polyallylamine hydrochloride building block represented by the following chemical formula (8) with n being not less than 3 nor more than 150.

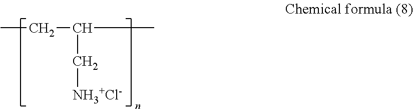

### (15) Fifteenth Aspect

Still in addition, in accordance with a fifteenth aspect of the present invention, there is provided a target recognition molecule according to any one of the aforesaid first, second, third, fifth and sixth aspects wherein the electrostatically-charged segment is a segment having a polydiallyldimethylammonium chloride building block represented by the following chemical formula (9) with n being not less than 3 nor more than 150.

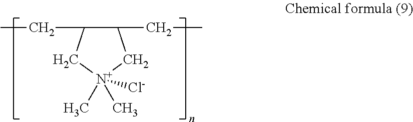

### (10) Sixteenth Aspect

Still in addition, in accordance with a sixteenth aspect of the present invention, there is provided a target recognition molecule according to any one of the aforesaid first, second, third, fifth and sixth aspects wherein the electrostatically-charged segment is a segment having a polyvinylpyridine building block represented by the following chemical formula (10) with n being nor more than 150.

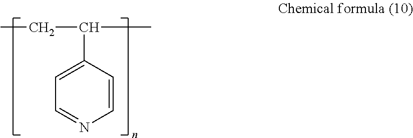

In the above chemical formulas 1 to 3 and 6 to 10, when the repeat unit (n) of the electrostatically-charged segment is increased, the synthesis cost increases. Moreover, when the repeat unit (n) is too increased (the length is too much), there occurs a folding or an entanglement of the molecular and so on, and thereby a specific sensitivity of the target recognition site is impaired. Therefore, the repeat unit (n) of the electrostatically-charged segment must be determined so that a specific sensitivity of the target recognition peptide segment may be obtained at a high level. For example, “n” of the electrostatically-charged segment is set to approximately 150 or less, 60 or less, or 20 or less.

### <Peptide-Based Electrostatically-Charged Segment>

### (17) Seventeenth Aspect

In accordance with a seventeenth aspect of the present invention, there is provided a target recognition molecule according to any one of the aforesaid first to third aspects wherein the electrostatically-charged segment is a segment having a peptide chain.

In this configuration, the target recognition molecule may be composed only of amino acids. Such a molecule is easy to prepare. As a method for preparing the target recognition molecule, either of the following methods may be used: the method in which the target recognition peptide segment and the electrostatically-charged segment are prepared separately and then both are bonded to each other; or the method in which both are continuously and integrally formed. In these methods, conventional techniques may be used. For example, as the method in which the target recognition molecule is continuously and integrally formed, a gene recombination technique may be used in which a DNA base sequence corresponding to the whole amino acid sequence of the target recognition molecule is synthesized and then this DNA base sequence is introduced into a target organism.

### (18) Eighteenth Aspect

Still in addition, in accordance with an eighteenth aspect of the present invention, there is provided a target recognition molecule according to the aforesaid seventeenth aspect wherein the peptide chain of the electrostatically-charged segment contains three or more acidic amino acid residues, one or more of which are selected from a group composed of an aspartic acid residue and a glutamic acid residue, and contains no basic amino acid residues such as an arginine residue and a lysine residue.

The electrostatically-charged segment according to this configuration is formed containing three or more particular acidic amino acid residues of low isoelectric point, and contains no basic amino acid residues so that it becomes strongly negatively charged in a solution from mild acidic to alkali. Therefore, the target recognition molecule having this configuration is suitable for the analysis of a target substance that employs a solution of from mild acidic to alkali.

The number of the acidic amino acid residues in the above configuration is preferably 4 or greater, more preferably 6 or greater, and still more preferably 8 or greater. And the number is preferably up to 30, and more preferably 20. The reason is as follows. A charge amount is increasing with the number of the acidic amino acid residues, while its synthesis cost is also increasing and further its handling becomes difficult due to a long chain of the molecule. Considering that stronger charge can be obtained from fewer linking units, it is preferable to continuously link the acidic amino acid residues consisting of 3 or greater units, preferably 5 or greater units and more preferably 8 or greater units.

### (19) Nineteenth Aspect

In accordance with a nineteenth aspect of the present invention, there is provided a target recognition molecule according to the aforesaid eighteenth aspect wherein a content rate in the number of the acidic amino acid residues is 60% or more in the peptide chain of the electrostatically-charged segment.

In the electrostatically-charged segment having the above configuration, no basic amino acid residues are contained and a content rate of neutral amino acids is less than 40%. Thus, since acidic amino acids are dominant, it is easy to adjust the charge of the electrostatically-charged segment to negative in the relations with pH of a solution.

### (20) Twentieth Aspect

In accordance with a twentieth aspect of the present invention, there is provided a target recognition molecule according to any one of the aforesaid seventeenth to nineteenth aspects wherein the average isoelectric point of the peptide chain constituting the target recognition peptide segment is 8 or less, and wherein the isoelectric point of the peptide chain constituting the electrostatically-charged segment is from 2.77 or more to 4.5 or less.

In this configuration, when a carrier solution having a pH around neutral is used, the target recognition peptide segment charges negatively or negligibly positively, or does not charge. On the other hand, the electrostatically-charged segment charges strongly negatively. This allows to preferentially draw the electrostatically-charged segment side to a positive electrode.

### (21) Twenty-First Aspect

In accordance with a twenty-first aspect of the present invention, there is provided a target recognition molecule according to any one of the aforesaid first to third aspects wherein the electrostatically-charged segment contains no basic amino acid residues such as an arginine residue and a lysine residue and contains 6 or more acidic amino acid residues, one or more of which are selected from a group composed of an aspartic acid residue and a glutamic acid residue; a content rate of the acidic amino acid residues is 60% or more in the peptide chain; and two or less neutral amino acid residues are interposed between adjacent two of the acidic amino acid residues.

In the above configuration, type (negative or positive polarity) and strength of the charge in the length direction cannot be extremely alternated. In addition, since there can be prepared the electrostatically-charged segment having sufficiently high negative charge density relative to a certain length, the electrostatically-charged segment properly serves as an immobilized site. Furthermore, in the above configuration, the number of neutral amino acid residues interposed between adjacent two of the acidic amino acid residues may be two, one or zero, but less neutral amino acid residues result in stronger charge density.

### (22) Twenty-Second Aspect

Still in addition, in accordance with an twenty-second aspect of the present invention, there is provided a target recognition molecule according to the aforesaid seventeenth aspect wherein the peptide chain of the electrostatically-charged segment contains three or more basic amino acid residues, one or more of which are selected from a group composed of an arginine residue and a lysine residue and contains no acidic amino acid residues such as an aspartic acid residue and a glutamic acid residue.

The electrostatically-charged segment according to this configuration is formed containing three or more particular basic amino acid residues of high isoelectric point, and contains no acidic amino acid residues so that it becomes strongly positively charged in a solution from mild alkali to acidic. Therefore, the target recognition molecule having this configuration is suitable for the analysis of a target substance that employs a solution of from mild alkali to acidic.

### (23) Twenty-Third Aspect

In accordance with a twenty-third aspect of the present invention, there is provided a target recognition molecule according to the aforesaid twenty-second aspect wherein a content rate in the number of the basic amino acid residues is 60% or greater in the peptide chain of the electrostatically-charged segment.

In the electrostatically-charged segment having the above configuration, no acidic amino acid residues contains and a content rate of neutral amino acid residues is less than 40%. Thus, since basic amino acid residues are dominant, it is easy to adjust the charge of the electrostatically-charged segment to positive in the relations with pH of a solution.

### (24) Twenty-Fourth Aspect

In accordance with a twenty-fourth aspect of the present invention, there is provided a target recognition molecule according to any one of the aforesaid seventeenth, twenty-second and twenty-third aspects wherein the average isoelectric point of the peptide chain constituting the target recognition peptide segment is 6 or greater, and wherein the average isoelectric point of the peptide chain constituting the electrostatically-charged segment is from 8 or greater to 10.76 or less.

In this configuration, when a carrier solution having a pH around neutral is used, the target recognition peptide segment charges positively or negligibly negatively, or does not charge. On the other hand, the electrostatically-charged segment charges strongly positively. This allows to preferentially draw the electrostatically-charged segment side to a negative electrode rather than the target recognition peptide segment side.

### (25) Twenty-Fifth Aspect

In accordance with a twenty-fifth aspect of the present invention, there is provided a target recognition molecule according to any one of the aforesaid first to third aspects wherein the electrostatically-charged segment contains no acidic amino acid residues such as an aspartic acid residue and a glutamic acid residue, and contains 6 or more basic amino acid residues, one or more of which are selected from a group composed of an arginine residue and a lysine residue; a content rate in the number of the basic amino acid residues is 60% or greater in the peptide chain of the electrostatically-charged segment; and two or less neutral amino acid residues are interposed between adjacent two of the basic amino acid residues.

In the above configuration, type (negative or positive polarity) and strength of the charge in the length direction cannot be extremely alternated. In addition, since there can be prepared the electrostatically-charged segment having sufficiently high positive charge density relative to a certain length, the electrostatically-charged segment properly serves as an immobilized site. Furthermore, in the above configuration, the number of neutral amino acid residues interposed between adjacent two of the basic amino acid residues may be two, one or zero, but less neutral amino acid residues is advantageous because higher charge density can be obtained.

The term “neutral amino acid” as used herein means an amino acid except for an acidic amino acid and a basic amino acid, and specifically includes alanine, asparagine, cysteine, glutamine, glycine, histidine, isoleucine, leucine, methionine, phenylalanine, proline, serine, threonine, tryptophan, tyrosine and valine.

### (26) Twenty-Sixth Aspect

In accordance with a twenty-sixth aspect of the present invention, there is provided a target recognition molecule according to any one of the aforesaid first to twenty-fifth aspects wherein the target recognition peptide segment contains a cysteine residue, and the electrostatically-charged segment is chemically linked to the sulfur atom in the cysteine residue.

### (27) Twenty-Seventh Aspect

In addition, in accordance with a twenty-seventh aspect of the present invention, there is provided a target recognition molecule according to any one of the aforesaid first to twenty-fifth aspects wherein either end of the target recognition peptide segment is a cysteine residue, and the electrostatically-charged segment is chemically linked to the sulfur atom in the cysteine residue.

### (28) Twenty-Eighth Aspect

Furthermore, in accordance with a twenty-eighth aspect of the present invention, there is provided a target recognition molecule according to any one of the aforesaid first to twenty-fifth aspects wherein the electrostatically-charged segment is chemically linked to the N-terminal or C-terminal of the amino acids constituting the target recognition peptide segment.

In this configuration, since the electrostatically-charged segment is linked at its end, the specificity of the target recognition peptide segment for the target substance is hardly inhibited.

### (29) Twenty-Ninth Aspect

In accordance with a twenty-ninth aspect of the present invention, there is provided a target recognition molecule according to any one of the aforesaid first to twenty-eighth aspects wherein the target recognition peptide segment is a peptide that contains 3 or more and 19 or less amino acid residues.

In the case of 3 or more and 19 or less amino acid, peptide synthesis can be easily performed, and a peptide that exerts target recognition ability can be obtained.

### (30) Thirtieth Aspect

In accordance with a thirtieth aspect of the present invention, there is provided a target recognition molecule according to any one of the aforesaid first to twenty-ninth aspects wherein further chemically linked to the electrostatically-charged segment is a base material immobilizing segment which is provided with a functional group for linkage to a base material.

When a base material immobilizing segment is further linked to the electrostatically-charged segment, the target recognition molecule can be efficiently and densely collected to an electrode forming portion of the base material due to electrical attraction. In this state, the target recognition molecule can be linked and immobilized to the electrode forming portion via the base material immobilizing segment. In a word, the target recognition molecule allows to realize a microfluidic device in which the molecule is densely immobilized at a predetermined site in the channel. In this microfluidic device, the target recognition molecule remains at the predetermined site in the channel even when voltage application is stopped.

### (31) Thirty-First Aspect

In accordance with a thirty-first aspect of the present invention, there is provided a method for immobilizing a target recognition molecule onto a surface of an electrode formed in a microchannel of a microfluidic device, wherein the method comprises: a target recognition molecule solution preparation step in which the target recognition molecule formed in accordance with the aforesaid fourth aspect is dissolved in a solution to thereby adjust pH of the solution to pH that is equal to or more than an average isoelectric point of the target recognition peptide segment, and a retaining step in which, with positive electric charges impressed to the electrode in the microchannel, the target recognition molecule containing solution is flowed in the microchannel so that the target recognition molecule in the solution is electrically absorbed and retained on the electrode surface.

### (32) Thirty-Second Aspect

In accordance with a thirty-second aspect of the present invention, there is provided a method for immobilizing a target recognition molecule onto a surface of an electrode formed in a microchannel of a microfluidic device, wherein the method comprises: a target recognition molecule solution preparation step in which the target recognition molecule formed in accordance with the aforesaid fifth aspect is dissolved in a solution to thereby adjust pH of the solution to pH that is equal to or less than an average isoelectric point of the target recognition peptide segment, and a retaining step in which, with negative electric charges impressed to the electrode in the microchannel, the target recognition molecule containing solution is flowed in the microchannel so that the target recognition molecule in the solution is electrically absorbed and retained on the electrode surface.

### (33) Thirty-Third

In accordance with a thirty-third aspect of the present invention, there is provided a method for immobilizing a target recognition molecule onto a surface of an electrode formed in a microchannel of a microfluidic device, wherein the method comprises: a target recognition molecule solution preparation step in which the target recognition molecule formed in accordance with the aforesaid sixth aspect is dissolved in a solution to thereby adjust pH of the solution to more than 6.5 and less than 7.5, and a retaining step in which, with either positive or negative electric charges impressed to the electrode in the microchannel, the target recognition molecule containing solution is flowed in the microchannel so that the target recognition molecule in the solution is electrically absorbed and retained on the electrode surface.

In the target recognition molecule according to the sixth aspect of the present invention, which is an essential component in the above configuration, its average isoelectric point is more than 6 and less than 8, and the three or more electrostatically-charged functions can charge negatively in a solution with pH 7.5 or more and can charge positively in a solution with pH 6.5 or less. Therefore, when pH of the target recognition molecule is adjusted to more than 6.5 and less than 7.5, charge strength of the electrostatically-charged segment can be made higher than that of the target recognition peptide segment and thus it becomes more easy to draw the side of the electrostatically-charged segment to the electrode.

### (34) Thirty-Fourth Aspect

In accordance with a thirty-fourth aspect of the present invention, there is provided a method for immobilizing a target recognition molecule onto a surface of an electrode formed in a microchannel of a microfluidic device, wherein the method comprises: a target recognition molecule solution preparation step in which the target recognition molecule formed in accordance with the aforesaid twentieth aspect is dissolved in a solution to thereby adjust pH of the solution to 6 or greater and 8.5 or less, and a retaining step in which, with positive electric charges impressed to the electrode in the microchannel, the target recognition molecule containing solution is flowed in the microchannel so that the target recognition molecule in the solution is electrically absorbed and retained on the electrode surface.

In this configuration, there may be used a carrier solution with pH around neutral, i.e. 6 or greater and 8.5 or less. In this carrier solution, the target recognition peptide segment may have no charge, a slight positive charge or a slight negative charge, while the electrostatically-charged segment has a strong negative charge. Therefore, the electrostatically-charged segment can be preferentially drawn to the electrode to which the positive charge is applied.

### (35) Thirty-Fifth Aspect

In accordance with a thirty-fifth aspect of the present invention, there is provided a method for immobilizing a target recognition molecule onto a surface of an electrode formed in a microchannel of a microfluidic device, wherein the method comprises: a target recognition molecule solution preparation step in which the target recognition molecule formed in accordance with the aforesaid twenty-fourth aspect is dissolved in a solution to thereby adjust pH of the solution to 6 or greater and 8.5 or less, and a retaining step in which, with negative electric charges impressed to the electrode in the microchannel, the target recognition molecule containing solution is flowed in the microchannel so that the target recognition molecule in the solution is electrically absorbed and retained on the electrode surface.

In this configuration, the target recognition peptide segment may have no charge, a slight positive charge or a slight negative charge, while the electrostatically-charged segment has a strong positive charge. Therefore, the electrostatically-charged segment can be preferentially drawn to the electrode to which the negative charge is applied.

### (36) Thirty-Sixth Aspect

In accordance with a thirty-sixth aspect of the present invention, there is provided a method for immobilizing a target recognition molecule onto a surface of an electrode formed in a microchannel of a microfluidic device, wherein the method comprises: a step for a target recognition molecule containing solution in which the target recognition molecule formed in accordance with the aforesaid thirtieth aspect is dissolved in an aqueous solvent to thereby adjust the solution to a predetermined pH; and a step in which, with electric charges having an opposite polarity to that of the electrostatically-charged segment in the target recognition molecule containing solution impressed to the electrode in the microchannel, the target recognition molecule containing solution is flowed in the microchannel so that the target recognition molecule in the solution is electrically absorbed and temporarily retained on the electrode surface, and then the target recognition molecule is immobilized on the electrode surface via a base material immobilizing segment of the target recognition molecule.

### (37) Thirty-Seventh Aspect

In accordance with a thirty-seventh aspect of the present invention, there is provided a target recognition molecule immobilization electrode plate comprising an electrode plate and the target recognition molecule as set forth in claim 3 which is immobilized on the electrode plate.

### (38) Thirty-Eighth Aspect

In accordance with a thirty-eighth aspect of the present invention, there is provided a particular molecule detection apparatus comprising a channel, an electrode plate disposed in the channel, and the target recognition molecule as set forth in claim 3 which is immobilized on the electrode plate.

The embodiment of the target recognition molecule according to the present invention is explained below and, through this explanation, the technical meaning of the configuration according to the present invention is clarified.

The target recognition molecule of the present invention has an enhanced property of assembling in the electric field, thereby making it possible to cause, by making use of such a property, target recognition molecules to assemble and be retained in a desired immobilization site. For example, in a microfluidic device with a microchannel formed therein, an electrode is formed at a desired location for the assembling and retaining of target recognition molecules. This is followed by application of a voltage to the electrode so that its surface charges positively or negatively. Under this state, a solution that contains the target recognition molecule (a target recognition molecule containing solution) in which target recognition molecules are dissolved is flowed through the microchannel, whereby the target recognition molecules can be trapped and held on the electrode surface by electrical action. This state is temporary immobilization, and can be released when voltage application is stopped.

For example, the target recognition molecule according to the second aspect of the present invention is provided with three or more electrostatically-charged functional groups that become electrically charged with charges of the same polarity in a solution and, in addition, has no functional groups that become electrically charged to different polarities. Therefore, if such a target recognition molecule is dissolved in a solution, the electrostatically-charged segment becomes electrically charged with charges of the same polarity. Accordingly, when a solution containing target recognition molecules of the present invention is flowed on the charge-applied immobilization site (electrode), target recognition molecules are attracted onto the electrode surface by electrostatic interaction and densely trapped there. This densely assembling state will be held as long as the electrode is electrically charged with charges. In a word, this method allows that the target recognition molecules are reversibly and densely held (temporarily immobilized) on the predetermined site in the microchannel.

For example, the following target recognition molecule (the target recognition molecule according to the twentieth aspect of the present invention) is exemplified: the target recognition peptide segment is composed of peptides with an average isoelectric point of 8 or less; the electrostatically-charged segment contains three or more acidic amino acid residues, one or more of which are selected from a group composed of an aspartic acid and a glutamic acid, and contains no basic amino acid residues such as an arginine residue and a lysine residue; and an average isoelectric point of the peptide chain making up the electrostatically-charged segment is pH 2.77 or more and 4.5 or less. When the above target recognition molecule is dissolved into a carrier solution with, for example, pH 7.5, the target recognition peptide segment has no charge, slight positive charge or slight negative charge while the electrostatically-charged segment has strong negative charge. And since the electrostatically-charged segment contains no basic amino acid residues, significant unevenness is not seen in its charge distribution.

When a carrier solution containing this target recognition molecule (target recognition molecule solution) is flowed into a channel providing for an electrode to which positive charge is applied, the electrostatically-charged segment (charging negatively) of the target recognition molecule is drawn to the electrode and retained on its surface. On the other hand, since the target recognition peptide segment of the target recognition molecule also has slight negative charge, each molecule is not electrically attached to other molecules in the solution.

According to the present invention, there can be provided the target recognition molecule in which the electrostatically-charged segment serves as a site retained on the electrode surface. When a solution containing this molecule is flowed on the electrode in which voltage application is properly controlled, only the electrostatically-charged segment in the target recognition molecule is retained on the electrode surface. In this state, since the electrostatically-charged segment serves as a spacer for maintaining the distance between the electrode surface and the target recognition peptide segment, the target recognition peptide segment can sway with the electrostatically-charged segment as a fixed end, and thereby can sufficiently exert specific recognition function that is its primary function. In addition, this molecule is trapped and held on the electrode disposed at a predetermined site in the channel (immobilizing site) with high efficiency and density, and further has reversible immobilization function that the immobilization is released when voltage application is stopped and the molecule becomes flowable.

In the target recognition molecules as explained above, the target recognition peptide segment may be directly linked to the electrostatically-charged segment, or a linkage member may be interposed between the target recognition peptide segment and the electrostatically-charged segment. In addition, a base material immobilizing segment may be added to the electrostatically-charged segment.

The target recognition molecule of the present invention is a chemical compound having a structure that it is provided, at one end thereof, with a target recognition peptide segment which specifically interacts with a target substance and at the other end with an electrostatically-charged segment which becomes either positively or negatively electrically charged. In the target recognition molecule of the present invention with this structure, the target recognition peptide segment exhibits a property of specifically recognizing a target substance as a target for analysis while the electrostatically-charged segment exhibits a property of densely assembling onto the applied electrode (immobilization site). Furthermore, the electrostatically-charged segment prevents the target recognition peptide segment from decreasing in the degree of freedom so that the target recognition peptide segment is allowed to function to sufficiently exert its specific recognition function.

Target recognition molecules according to the present invention, when used, are efficiently and densely held in an immobilization site where the electrode is formed, and such immobilization by electric hold is reversible, thereby achieving significant improvement in the usability of microfluidic devices. In addition, the dense immobilization of target recognition molecules can significantly improve the analytical sensitivity and precision of microfluidic devices.

Furthermore, in the target recognition molecule of the present invention in which a base material immobilizing segment with a function group for linkage to a base material is linked to the electrostatically-charged segment, it is possible that target recognition molecules are first densely brought together by applying a voltage to a site that requires immobilization and, in this state, the target recognition molecules and the base material are linked together via the base material immobilizing segment, and thus the dense immobilization is available. The linkage via the base material immobilizing segment is not released even if the voltage application is stopped, thereby dense and irreversible immobilization can be achieved.

### BRIEF DESCRIPTION OF THE DRAWINGS

For a more complete understanding of the present invention and the advantages thereof, reference is now made to the following descriptions taken in conjunction with the accompanying drawings, in which:

FIG. 1 is a conceptual diagram showing a connection of constituent elements of a target recognition molecule of the present invention.

FIG. 2 is a conceptual diagram showing a state of how a target recognition molecule shown in FIG. 1 is held by intercharge interaction onto an electrode (immobilization site).

FIG. 3 is a conceptual diagram showing a connection of constituent elements of a target recognition molecule of the present invention which has a base material immobilizing segment.

FIG. 4 is a conceptual diagram showing a state of how a target recognition molecule of the present invention which has a base material immobilizing segment is held by intercharge interaction onto an electrode (immobilization site).

FIG. 5 is a conceptual diagram showing a condition of how a target recognition molecule of the present invention which has a base material immobilizing segment is chemically linked to an electrode as a base material.

FIG. 6 is a diagram showing an example of an analytical apparatus which is an application target of a target recognition molecule of the present invention.

### DETAILED DESCRIPTION OF THE INVENTION

Examples for carrying out the present invention will be described successively hereinafter.

### First Group of Examples

### Example 1-1

Example 1-1 is an example of a target recognition molecular in which both a target recognition peptide segment and an electrostatically-charged segment are composed of peptides.

### (Target Recognition Peptide Segment)

As a target recognition peptide segment, there was prepared a protein kinase B (PKB) substrate peptide. This peptide has an amino acid sequence (G-R-P-R-T-S-S-F-A-E-G) and each serine residue is phosphorylated. In addition, the average isoelectric point of the PKB substrate calculated based on the following Table 1 and mathematical formula (1) is 6.5.

$$Mathematical formula ( 1 )$$

$$\begin{matrix}\begin{matrix}ISOELECTRIC POINT \\ OF TARGET\end{matrix} \\ RECOGNITION PS\end{matrix} = \frac{\begin{matrix}\begin{matrix}SUM OF ISOELECTRIC POINTS OF \\ AMINO ACIDS CORRESPONDING\end{matrix} \\ RESPECTIVELY TO AMINO ACID RESIDUES\end{matrix}}{NUMBER OF AMINO ACID RESIDUES} \qquad$$

TABLE 1

| amino acid class | abbrev. Form | isoelectric point |
|---|---|---|
| alanine | A (Ala) | 6.00 |
| arginine | R (Arg) | 10.76 |
| asparagine | N (Asx) | 5.41 |
| aspartic acid | D (Asp) | 2.77 |
| cysteine | C (Cys) | 5.05 |
| glutamine | Q (Glx) | 5.65 |
| glutamic acid | E (Glu) | 3.22 |
| glycine | G (Gly) | 5.97 |
| histidine | H (His) | 7.59 |
| isoleucine | I (Ile) | 6.05 |
| leucine | L (Leu) | 5.98 |
| lysine | K (Lys) | 9.75 |
| methionine | M (Met) | 5.74 |
| phenylalanine | F (Phe) | 5.48 |
| proline | P (Pro) | 6.30 |
| serine | S (Ser) | 5.68 |
| threonine | T (Thr) | 6.16 |
| tryptophan | W (Trp) | 5.89 |
| tyrosine | Y (Tyr) | 5.66 |
| valine | V (Val) | 5.96 |

### (Electrostatically-Charged Segment)

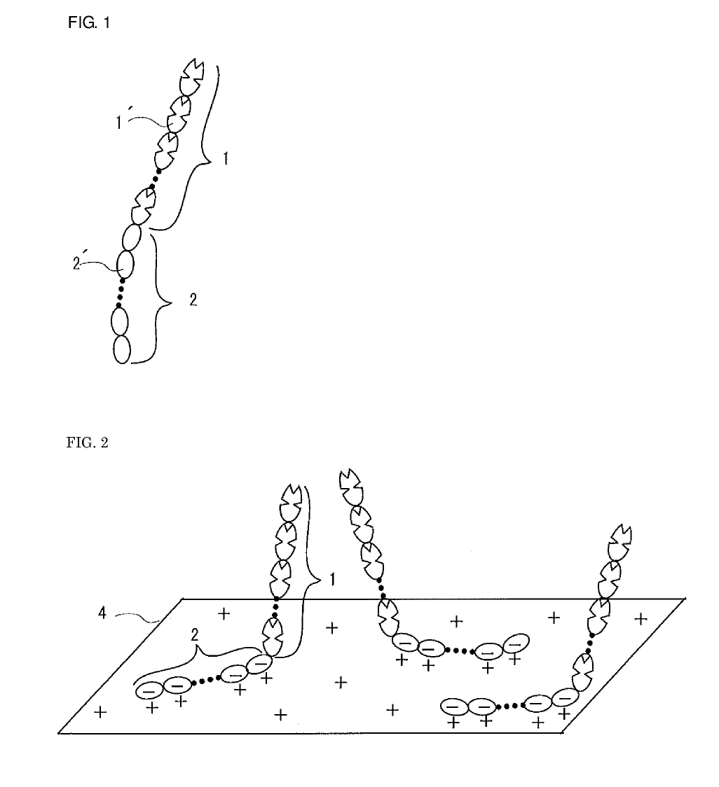

As an electrostatically-charged segment, there was used a peptide (amino acid sequence; DDDDDDDD) comprised of a coupled series of eight aspartic acids (D) which are acidic amino acids. This electrostatically-charged segment has an average isoelectric point of 2.77, and is hydrophilic.
The amino acid composition of the target recognition molecule according to Example 1-1 is shown in the chemical formula 11, and its conceptional structure is shown in FIG. 1. This target recognition molecule itself has an average isoelectric point of 4.95.

G-R-P-R-T-S-S-F-A-E-G-D-D-D-D-D-D-D-D Chemical formula (11);

There is explained below a method for preparing this target recognition molecule.

(1) There was prepared a commercially available amino acid in which all function groups except for an α-carboxyl group (an α-amino group and side chain groups) are protected. And the α-amino group of this amino acid is protected with Fmoc (fluorenyl-methoxy-carbonyl group); the side chain calboxyl group of aspartic acid is protected with cyclohexyl ester; the guanidino group of arginine is protected with p-toluenesurfonic acid; and the OH group of serine is protected with dimethyl phosphate.

(2) A carboxyl group of an amino acid (aspartic acid) serving as a C-terminal was immobilized to polystyrene (a support) whose surface is modified with an amino group.

(3) The support resulting from the above (2) to which aspartic acid was immobilized was mixed with 20% of piperidine/N,N-dimethylformamide (DMF), and thereby the amino group protected with Fmoc was deprotected.

(4) A by-product resulting from the deprotection was washed and removed.

(5) Aspartic acid (an amino acid to be linked to the amino acid serving as the C-terminal), 1-hydroxybenzotriazole (HOBt) and diisopropyl carbodiimide (DIC) were dissolved into N-methylpyrrolidone (NMP) so that the concentrations of aspartic acid, 1-hydroxybenzotriazole and diisopropyl carbodiimide were 0.5 M (“M” is hereafter used as an abbreviation for “mol/liter”.), 1.1 M and 1.1 M, respectively.

(6) The solution resulting from the above (5) was mixed with the solution obtained after washing of the above (4). Thereby, a condensation reaction occurred between the amino group of one aspartic acid (D) and the carboxyl group of another aspartic acid (D) to form a peptide bond.

(7) Amino acids that had not been peptide-bonded in the above (6) was washed and removed.

(8) The third and later amino acids from the C-terminal were sequentially linked in the similar way to the above (5) to (7). And finally, the amino acid chain was extended to glycine (G) having the N-terminal.

(9) After the extension process of the amino acid chain, the completed chain was treated with a mixture solution of trifluoroacetic acid (TFA) containing 3 vol. % of triisopropylsilane and 2 vol. % of pure water in order to deprotect the protect groups of the side chain (two methyl groups of dimethyl phosphate in the case of serine) in each amino acid residue constituting the amino acid chain. Thus, the target recognition molecule according to this Example was prepared.

Regarding preparation of the target recognition peptide segment and electrostatically-charged segment, whole segment including both segments may be also prepared by the biological method using gene recombination. In this case, since phosphorylation of serine is required in the target recognition molecule, it is preferable to introduce a gene into a eukaryotic organism such as yeast.

FIG. 1 shows a conceptual structure based on the functions of the target recognition molecule. Referring to FIG. 1, the reference numeral 1 denotes a target recognition peptide segment; the reference numeral 2 denotes an electrostatically-charged segment; the reference numeral 1′ denotes an amino acid residue as a building block of the target recognition peptide segment; and the reference numeral 2′ denotes a building block of the electrostatically-charged segment (an amino acid residue in this example). In addition, what is meant by “a series of dots” (i.e. “• • •” shown in the figure) is an omission of building blocks.

The target recognition molecule is dissolved in water, and if the solution is at a pH from mildly basic to neutral (e.g. PH 7.3), the electrostatically-charged segment portion becomes strongly negatively charged, and the target recognition peptide segment becomes slightly positively charged. Therefore, upon contact of this solution with the surface of a positively charged electrode, the electrostatically-charged segment portion is electrically held and immobilized on the electrode surface, while the target recognition peptide segment is not directly bound to the electrode. This aspect is shown in FIG. 2. FIG. 2 is a conceptual scheme showing an aspect of target recognition molecules being held on the electrode surface. The reference numeral 4 in FIG. 2 refers to a base material (electrode).

Next, making reference to FIG. 6, an example of how the target recognition molecule is used will be described. FIG. 6 illustrates an analytical apparatus 10 which employs an analytical microfluidic device, and the reference numeral 11 denotes a solution inlet; the reference numeral 12 denotes a microchannel; the reference numeral 13 denotes an outlet; the reference numerals 14 and 15 denote a pair of electrodes; and the reference numeral 16 denotes a detector. The basic procedure of an analytical method with the aid of this analytical apparatus is as follows.

The target recognition molecule of Example 1-1 is dissolved in a carrier liquid composed of a phosphate buffered saline having a pH value of, for example, 7.3. The concentration is, for example, 100 ug/mL.

Next, with a direct current voltage (for example, from 1 to 10 V) impressed on the electrodes in pair (either one of the surfaces of the electrodes serves as an immobilization part), the target recognition molecule-containing carrier liquid is poured from the solution inlet 11 to flow through the inside of the microchannel 12. The target recognition molecule of Example 1-1 is attracted and immobilized onto the electrode 14 because the electrostatically-charged segment becomes negatively charged in the solution having a pH value of 7.3, as described above. In this state, the inside of the microchannel is cleansed by the aforesaid carrier liquid (containing no target recognition molecules). This completes an operation for immobilizing the target recognition molecule.

Thereafter, when a carrier solution of pH 7.3 containing a target substance (a target) is flowed, the target substance is captured at the target recognition peptide segment. The operation after immobilization may be based on a known analytical technique, e.g. a non-labeled immunoassay method or a labeled immunoassay method (for example, a sandwich assay method). In addition, it is possible to use, for example, a thermal lens, a surface plasmon resonance sensor, or a crystal oscillator as a detector and, in addition, it is also possible to use an electrode (immobilization part) itself as an electrochemical detector.

The voltage application is stopped at the stage in which the target recognition molecule does not have to be held on the electrode surface anymore (for example, the stage when the analysis is completed), and then a cleaning solution is flowed. Since the immobilization to the electrode is released as soon as the voltage application stops, the target recognition molecule is flowed out of the channel system by the cleaning carrier solution. Thereby, reuse of the microfluidic device can be realized. In the cleaning, when pH of the cleaning solution is set to pH suitable for cleaning on the basis of the charge of target recognition molecule, the cleaning effect is further enhanced.

The target recognition molecule according to Example 1-1 is composed of only peptides. However, only the electrostatically-charged segment is immobilized on the electrode while the target recognition peptide segment is not immobilized and freely sways in the carrier solution. For this reason, the immobilization does not significantly impair the target recognition function (target trap function).

In addition, as a material to form the electrode 14, for example, metals such as gold (Au), copper (Cu), silver (Ag), platinum (Pt) and so on or electrically conductive plastics can be used. And the electrode may be preformed, for example, by applying such a material to a site for immobilization during the preparation of a microfluidic device.

### Example 1-2

A cysteine (C) is introduced, as a base material immobilizing segment, to the C-terminal of the electrostatically-charged segment (amino acid sequence; DDDDDDDD) of the target recognition molecule of Example 1. The chemical formula (12) shows a target recognition molecule of Example 1-2. The molecule of the chemical formula (12) has an average isoelectric point of 4.90. This target recognition molecule can be prepared in the similar manner to the above Example 1-1.

G-R-P-R-T-S-S-F-A-E-G-D-D-D-D-D-D-D-D-C Chemical formula (12):

The target recognition molecule of Example 1-2 has such a property that it can be chemically linked, via the thiol group (elemental sulfur) of a cysteine residue, to the surface of the gold electrode. Therefore, with the gold electrode being electrically charged, a target recognition molecule containing solution is flowed, whereby target recognition molecules are densely brought together on the surface of the gold electrode and they are chemically linked to the surface of the gold (Au) electrode. After once chemically linked to the electrode surface, the immobilization state is retained even when the voltage application to the electrode is stopped.

### Example 1-3

In Example 1-2, a (N-[4-(p-Azidosalicylamido) butyl]-3′-(2′-pyridyldithio) propionamide) (APDP; produced by Thermo Corporation) was further reacted with the thiol group of the cysteine residue in order to introduce an azido group which is a photocrosslinking group into the terminal. A disulfide bond of the aforesaid APDP and an SH group of the cysteine are reacted (disulfide exchange) and linked together.

The chemical formula (13) shows the structure of a target recognition molecule of Example 1-3.

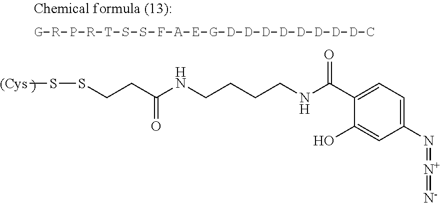

In the chemical formula (13) as shown above, the portion after this including a cysteine residue serves as a base material immobilizing segment. Further, in this example, it may be possible to arrange that, since the electrostatically-charged segment is composed of acidic amino acids and the cysteine is also an acidic amino acid, the cysteine-containing sequence (DDDDDDDD-C) is recognized as an electrostatically-charged segment while the portion after the S of the cysteine residue linked to the photocrosslinking group (azido group) can be set as a base material immobilizing segment.

Since, for the case of the target recognition molecule of Example 1-3, the base material immobilizing segment has a photocrosslinking group (azido group), this makes it possible to bring the target recognition molecule and the base material into chemical linkage (immobilization) by irradiation of the based material surface with light beams of UV long wavelength.

### Example 1-4

By use of an N-(6-Maleimidocaproyloxy) succinimide (Dojindo Laboratories) as a substitute for the (N-[4-(p-Azidosalicylamido) butyl]-3′-(2′-pyridyldithio) propionamide) of Example 1-3, a succinimide group was introduced into the thiol group of the cysteine residue.

A target recognition molecule according to this example has a succinimide group at its molecular end so that it can be brought into chemically linkage (immobilization) onto the base material surface having an amino group.

As a process for preparing the surface of a base material with amino groups, there is exemplified a method in which a thin film of gold is formed on a base plate and then a SAM (Self-assembled monolayer) film having an amino terminal is formed on the gold thin film by use of 11-Amino-1-undecanethiol, hydrochloride (Dojindo Laboratories).

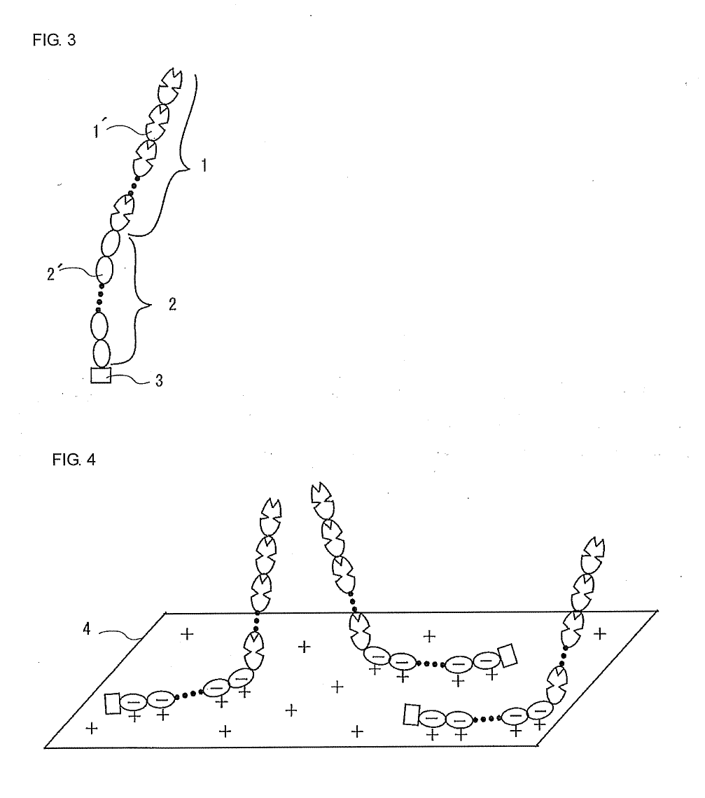

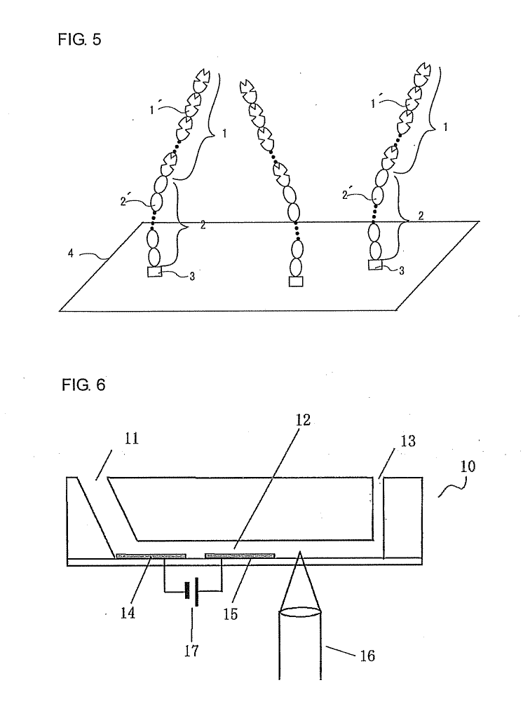

Referring to FIG. 3, there is shown a conceptual structure for the target recognition molecules of Examples 1-2 to 1-4. In addition, FIG. 4 shows an aspect of these target recognition molecules being electrostatically adsorbed and immobilized onto the electrically charged electrode surface (a base material surface). Further, FIG. 5 shows an aspect of the target recognition molecules being chemically linked, through their respective base material immobilizing segments, to the base material surface and uprising in the solution after stopping the voltage application to the electrode surface.

As shown in FIGS. 3-5, for the case of the target recognition molecules of Examples 1-2 to 1-4, molecules are brought together at the electrode by electrostatic attraction force and in this state, the functional group of each base material immobilizing segment can be linked to the electrode surface. It is therefore possible to accomplish immobilization with high efficiency and high strength. After the immobilization, the immobilization state will be retained even when the electrical current to the electrode is disconnected.

### Second Group of Examples

### Example 2-1

### Target Recognition Peptide Segment

As a target recognition peptide segment, there was prepared a protein kinase A (PKA) substrate peptide. This peptide has an amino acid sequence (L-R-R-A-S-L-G) and its serine residue is phosphorylated. In addition, the average isoelectric point of the PKA substrate calculated based on Table 1 and the mathematical formula (1) is 7.3.

### [Electrostatically-Charged Segment]

On the other hand, as an electrostatically-charged segment, there was used a peptide (SEQ; R-R-R-R-R-R-R-R-R-R) resulting from linking together ten arginines. The target recognition molecule was prepared in the similar manner to the above Example 1-1. This target recognition molecule of Example 2-1 is shown in the chemical formula (14). The molecule of the chemical formula (14) has an average isoelectric point of 9.34.

L-R-R-A-S-L-G-R-R-R-R-R-R-R-R-R-R Chemical formula (14):

Since the electrostatically-charged segment in this target recognition molecule has a high average isoelectric point, there is used a carrier solution having pH lower than the average isoelectric point of the electrostatically-charged segment.

As a basic amino acid that constitutes the electrostatically-charged segment, lysine and arginine may be used. As an acidic amino acid, aspartic acid and glutamic acid may be used. For example, even if only lysine or a combination of lysine and arginine is used as amino acid components of the electrostatically-charged segment in the molecule of Example 2-1 in place of arginine, a target recognition molecule exerting the similar function is obtained. Furthermore, even if the electrostatically-charged segment contains a neural amino acid, it is possible to obtain a target recognition molecule having both a target recognition function and an immobilizing function.

However, a high content of neutral amino acids decreases charge intensity and density of the electrostatically-charged segment. Therefore, it is preferable that the electrostatically-charged segment contains 6 or more basic amino acid residues; a content rate in the number of the basic amino acid residues is 60% or greater; and two or less neutral amino acid residues are interposed between adjacent two of the basic amino acid residues. An example of such a molecule is shown in the below expansion example. On the other hand, in the case of the electrostatically-charged that mainly contains acidic amino acids, it is preferable that the above basic amino acid is replaced to an acidic amino acids (aspartic acid and glutamic acid) and then neutral amino acids is limited in the similar way.

### Expansion Example

[L-R-R-A-S-L-G]-[R-R-R-A-H-K-K-K-T-R-K-R-P-K]

### Example 2-2

As with the above Example 1-2, a cysteine (C) is introduced, as a base material immobilizing segment, to the C-terminal of the electrostatically-charged segment (amino acid sequence; RRRRRRRRRR) of the target recognition molecule. This target recognition molecule can be also prepared in the similar manner to the above Example 1-1. The chemical formula (15) shows the target recognition molecule of Example 2-2. The molecule of the chemical formula (15) has an average isoelectric point of 9.10.

L-R-R-A-S-L-G-R-R-R-R-R-R-R-R-R-R-C Chemical formula (15):

### Example 2-3

A target recognition molecule of Example 2-3, in which a photocrosslinking group (an azido group) was introduced into the terminal of the molecule, was prepared in the similar manner to the above Example 1-3. The target recognition molecule of Example 2-3 is shown in the chemical formula 16.

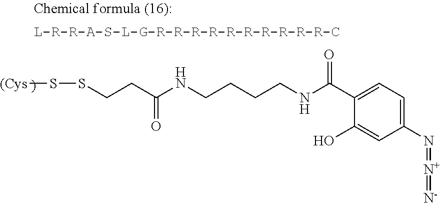

### Example 2-4

A target recognition molecule of Example 2-4, in which a succinimide group was introduced into the thiol group of the cysteine residue, was prepared in the similar manner to the above Example 1-4. The target recognition molecule of Example 2-4 is shown in the chemical formula 17.

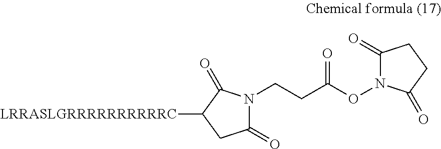

### Third Group of Examples

### Example 3

As a target recognition peptide segment, there was prepared a protein kinase A (PKA) substrate peptide (SEQ: L-R-R-A-S-L-G), and a lysine residue (K) was introduced to the C-terminal of the peptide. On the other hand, as an electrostatically-charged segment, there was used a segment that has a polyacrylic acid building block (n=14, R=Na) as shown in the following chemical formula (1).

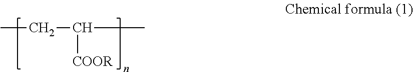

wherein R is H, Na, or K.

Some of carboxyl groups in the electrostatically-charged segment were activated with 1-ethyl-3-[3-dimethylaminopropyl]carbodiimide hydrochloride (Thermo Corporation), and were linked to the side chain amino group of the lysine residue in the target recognition peptide segment.

One of the structures of the target recognition molecule of Example 3 is shown in the chemical formula (18).

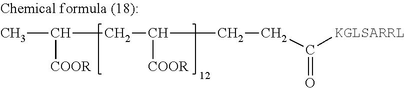

wherein R is H, Na, or K.

### Fourth Group of Examples

### Example 4

As a target recognition peptide segment, there was used a PKA substrate peptide (SEQ: LRRASLG) of the same type as used in the foregoing first group of examples, and a cysteine residue was introduced to the C-terminal of the peptide.

As an electrostatically-charged segment, there was used a segment that has polyethylenimine building blocks (n=14) as shown in the following the chemical formula (7). Some of amino groups in the electrostatically-charged segment were reacted with succinimide groups in N-(a-Maleimidoacetoxy) succinimide ester (Thermo Corporation). In addition, a maleimide moiety was linked to the thiol moiety of the cysteine residue, in the target recognition peptide segment.

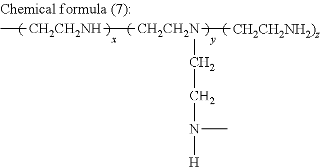

wherein x: y: z=0.5: 0.25: 0.25 and [x+y+z] is an integer not less than 3 nor more than 150.

A structure of the target recognition molecule of Example 4 is shown in chemical formula 19.

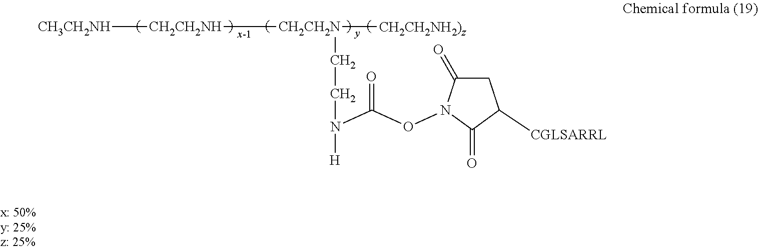

### Fifth Group of Examples

### Example 5

As a target recognition peptide segment, there was used a PKA substrate peptide (SEQ: L-R-R-A-S-L-G) of the same type as used in the foregoing first group of examples, and a lysine residue (K) was introduced to the C-terminal of the peptide.

As an electrostatically-charged segment, there was used a segment that has poly-diallyldimethylammonium chloride building blocks (n=14) shown in the chemical formula (9). In addition, an acrylic acid building block for linkage to the target recognition peptide segment was introduced into the electrostatically-charged segment, and a carboxylic group of the acrylic acid was linked to a side chain amino group of the lysine.

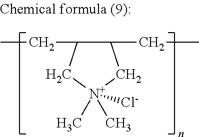

A structure of the target recognition molecule of Example 5 is shown in the chemical formula (20).

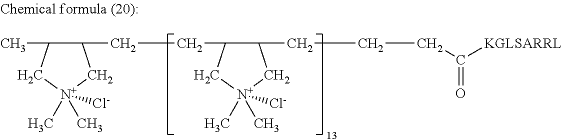

### Sixth Group of Examples

### Example 6

In Example 5, as an electrostatically-charged segment, there was used a segment that has, as a substitute for poly-diallyldimethylammonium chloride, polyallylamine building blocks (n=14) shown in the chemical formula (8). In addition, an acrylic acid building block for linkage to the target recognition peptide segment was introduced into the electrostatically-charged segment. With this exception, a target recognition molecule according to Example 6 was prepared in the same way as Example 5. A structure of this molecule is shown in the chemical formula (21).

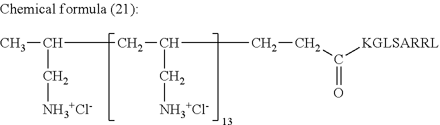

### Seventh Group of Examples

### Example 7

As a target recognition peptide segment, there was used a PKA substrate peptide (SEQ: LRRASLG) of the same type as used in the foregoing first group of examples, and a lysine residue (K) was introduced to the C-terminal of the peptide.

As an electrostatically-charged segment, there was used a segment that has polyvinylpyridine building blocks (n=14) shown in the chemical formula (10). In addition, an acrylic acid building block for linkage to the target recognition peptide segment was introduced into the electrostatically-charged segment. With this exception, a target recognition molecule according to Example 7 was prepared in the same way as Example 5.

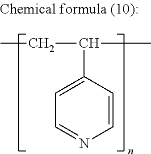

A structure of the target recognition molecule of Example 7 is shown in the chemical formula (22).

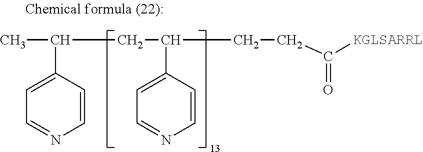

### Eighth Group of Examples

### Example 8

As a target recognition peptide segment, there was used a PKA substrate peptide (SEQ: LRRASLG) of the same type as used in the foregoing first group of examples, and a lysine residue (K) was introduced to the terminal of the peptide.

As an electrostatically-charged segment, there was used a segment that has an octonucleotide (one chain of which is a poly-deoxyadenosine-monophosphate and the other chain (complementary chain) of which is a poly-deoxythymidine-monophosphate) with a (CH₂)₆SH introduced into a 5′-terminal phosphoric acid of the one chain (see the chemical formula (23)).

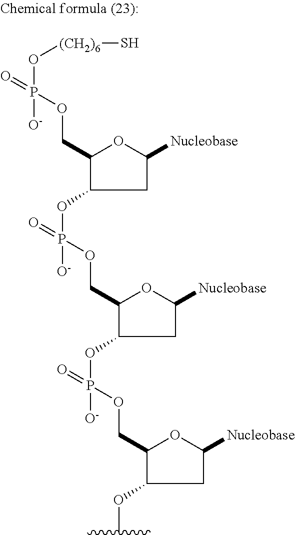

By using N-(6-Maleimidocaproyloxy) succinimide (Dojindo Laboratories), a succinimide group was introduced to the thiol group in the electrostatically-charged segment, and reacted with an amino group in the lysine residue of the target recognition peptide segment. A structure of the target recognition molecule of Example 8 is shown in the chemical formula (24).

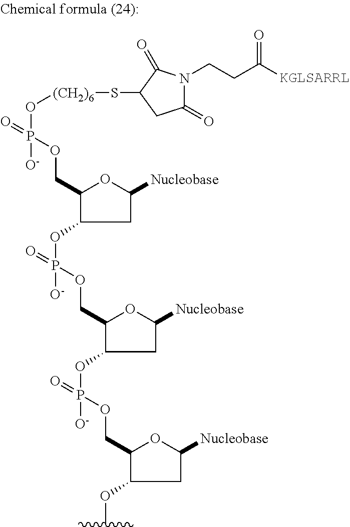

### <Complement>

In the above Example 3, it is possible to use, in place of an electrostatically-charged segment having a polyacrylic acid building block as described above, an electrostatically-charged segment having either a polystyrene sulfonic acid building block as shown in the chemical formula (2) or a polyvinyl sulfate building block as shown in the chemical formula (3).

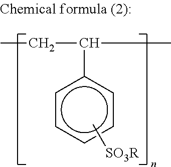

wherein R is H, Na, or K.

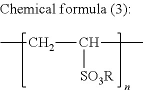

wherein R is H, Na, or K.

In addition, the target recognition molecules without a base material immobilizing segment are shown in Examples 3 to 9. However, as described in the first group of Examples, these molecules may be chemically linked to a base material immobilizing segment.

The target recognition molecule according to the present invention may have a linking element that intervenes between the target recognition peptide segment and the electrostatically-charged segment. As the linking element, for example, polyethylene glycol shown in the chemical formula (25) may be used.

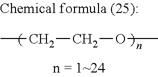

### <Length of the Electrostatically-Charged Segment>

If the length (arm length) of the electrostatically-charged segment is too long, this causes disadvantages such as an intermolecular entanglement. On the other hand, if the length of the electrostatically-charged segment is too short, this results in a reduced degree of freedom of the target recognition segment. Therefore, it is required that the length of the electrostatically-charged segment be properly selected in relation to its own properties as well as in relation to the target recognition segment. Preferably, the length of the electrostatically-charged segment is equal to or more than that of the target recognition peptide segment. And it is more preferable that the length of the electrostatically-charged segment is from once to twice the length of the target recognition peptide segment. In addition, if the repeat unit (n) is less than 3, this is undesirable because the force of attraction by electrostatic interaction becomes deficient. Therefore, three or more repeat units (n) are preferable, and three or more and 150 or less repeat units (n) are more preferable.

### <Average Isoelectric Point of the Target Recognition Peptide Segment>

In a microfluidic device using a target recognition molecule, there is usually used a carrier solution (an aqueous solution) having a near-neutral pH value (pH value=about 7±1). Since the average isoelectric point of each of the target recognition peptide segments of the foregoing Example 2 is 7.3, the electric charge of their target recognition peptide segment part reaches a negligible level if the target recognition molecule according to each of the examples is solved in a neutral carrier solution (pH value=about 7±1). In other words, assuming a neutral carrier solution (pH value=about 7±1), since the target recognition peptide segment part having the average isoelectric point of 7.3 gives only a little influence, when the target recognition peptide segment is added to an electrostatically-charged segment having functional groups with either strong positive or negative charge in the above pH range, the behavior of the whole molecule can be electrostatically controlled.

However, when the average isoelectric point of the target recognition peptide segment is close to that of the electrostatically-charged segment, the whole molecule is contacted and immobilized to the electrode. The immobilization of the whole molecule may impair target recognition function of the target recognition peptide segment. Therefore, it is desirable that the target recognition peptide segment charges with opposite polarity to the electrostatically-charged segment in a solution having the same pH. As the above “a solution having the same pH”, a solution having pH around neutral is desired. The reason is that the target recognition peptide segment is denatured due to the property of peptides when a strong acid or basic solution is used.

Specifically, when the target recognition peptide segment in the molecule in which both the target recognition peptide segment and the electrostatically-charged segment are composed of peptides has an average isoelectric point of “8 or less” (preferably “6 or more and 7.5 or less”), an average isoelectric point of the electrostatically-charged segment is set to “2.77 or more and 4.5 or less”. When such a target recognition peptide segment complying with the above condition is dissolved in a carrier solution (buffer aqueous solution) with, for example, pH around 7, the target recognition peptide segment has slight positive or negative charge or no charge while the electrostatically-charged segment has strong negative charge. Since the target recognition peptide segment in the molecule has only a small impact on the charge, only the electrostatically-charged segment can be held on the electrode (immobilizing site) in a preferable manner by properly controlling positive voltage application.

On the other hand, when the target recognition peptide has an average isoelectric point of “6 or more”, preferably “6.5 or more and 8 or less”, an average isoelectric point of the electrostatically-charged segment is set to “8 or more”, preferably “8.5 or more and 10.76 or less” and more preferably “9.5 or more and 10.76 or less”. When such a target recognition peptide segment complying with the above condition is dissolved in a carrier solution (buffer aqueous solution) with, for example, pH around 7, the target recognition peptide segment has slight positive or negative charge or no charge while the electrostatically-charged segment has strong positive charge. Since the target recognition peptide segment in the molecule has only a small impact on the charge, only the electrostatically-charged segment can be held on the electrode (immobilizing site) in a preferable manner by properly controlling negative voltage application.

### <Selection of the Target Recognition Peptide Segment>

Each of the foregoing examples uses a protein kinase A substrate peptide or a protein kinase B substrate peptide as a target recognition peptide segment. However, the target recognition peptide segment as a main element of the present invention is not limited to the aforesaid substances. The target recognition peptide segment according to the present invention may be any peptide as long as it can specifically recognize a target substance. Whether or not it is a peptide that specifically recognizes a target substance is determined in relation to a target substance as a detection object.

A method for select the target recognition peptide segment includes known technologies, such as phage display technology (Phage Display—Laboratory Manual. Cold Spring Harbor Laboratory Press, 2001, Barbas. C. et al.) and spot synthesis technology (The SPOT-synthesis technique. Synthesis peptide arrays on membrane supports-principles and applications. J. Immunol. Methods, 267, 2002, 13-26, R. Frank). These methods allow to determine an amino acid sequence of the peptide that can recognize a target substance.

The material of the peptide of the target recognition peptide segment may be either naturally derived or artificially synthesized, and there is no limitation regarding the process of peptide synthesis.

### INDUSTRIAL APPLICABILITY

The target recognition molecule of the present invention is a novel chemical molecule including a target recognition segment as a binding site which specifically interacts with a target substance and an electrostatically-charged segment which is provided with an electrostatic property. The use of a solution containing a target recognition molecule of the present invention makes it possible that such target recognition molecules can be densely brought together in a charge-applied immobilization site in a self-assembly manner and reversibly immobilized there. In addition, the use of a target recognition molecule of the present invention which is provided with a base material immobilizing segment makes it possible that such target recognition molecules can be densely brought together in a charge-applied immobilization site in a self-assembly manner and immobilized there. In an analytical field or medical field in which antigen-antibody reaction is used, these target recognition molecules of the present invention improve the usability, and reliability for assay accuracy and reproductivity of microfluidic devices, and contribute to the development in therapeutic techniques. Therefore, the industrial applicability of the target recognition molecules of the present invention is high.

### REFERENCE SIGNS LIST

1 Target recognition peptide segment
1′ Target recognition peptide segment building block
2 Electrostatically-charged segment
2′ Electrostatically-charged segment building block
3 Base material immobilizing segment
4 Base material (Electrode)
10 Analytical apparatus
11 Liquid inlet port
12 Microchannel
13 Outlet port
14, 15 Electrodes (Either one of them serves as an immobilization site.)
16 Detector
17 Power supply

What is claimed is:

1. A target recognition molecule comprising:
a target recognition peptide segment having an amino acid sequence which specifically interacts with a target substance capable of causing an immune reaction; and
an electrostatically-charged segment which does not specifically interact with the target substance and which is provided with three or more electrostatically-charged functional groups capable of being electrically charged with charges of the same polarity in the same solution.

2. The target recognition molecule as set forth in claim 1,
wherein an electrostatically-charged segment which is not provided with any functional groups capable of being electrically charged with charges of a different polarity in the same solution.

3. The target recognition molecule as set forth in claim 1,
wherein the electrostatically-charged segment is directly linked to an amino acid constituting the target recognition peptide segment.

4. The target recognition molecule as set forth in claim 3,
wherein an average isoelectric point of the target recognition peptide segment is 6 or less, and the three or more electrostatically-charged functional groups of the electrostatically-charged segment can charge negatively in a solution of pH 6.5 or more.

5. The target recognition molecule as set forth in claim 3,
wherein an average isoelectric point of the target recognition peptide segment is 8 or more, and the three or more electrostatically-charged functional groups of the electrostatically-charged segment can charge positively in a solution of pH 7.5 or less.

6. The target recognition molecule as set forth in claim 3,
wherein an average isoelectric point of the target recognition peptide segment is more than 6 and less than 8, and the three or more electrostatically-charged functional groups of the electrostatically-charged segment can charge negatively in a solution of pH 6.5 or more, or positively in a solution of pH 7.5 or less.

7. The target recognition molecule as set forth in claim 3,
wherein the electrostatically-charged segment is a segment which has a polyacrylic acid building block represented by the following chemical formula (1) with n being not less than 3 nor more than 150. 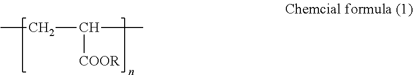
wherein R is H, Na, or K.

8. The target recognition molecule as set forth in claim 3,
wherein the electrostatically-charged segment is a segment having a polystyrene sulfonic acid building block represented by the following chemical formula (2) with n being not less than 3 nor more than 150. 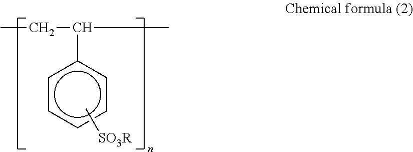
wherein R is H, Na, or K.

9. The target recognition molecule as set forth in claim 3,
wherein the electrostatically-charged segment is a segment having a polyvinyl sulfate building block represented by the following chemical formula (3) with n being not less than 3 nor more than 150. 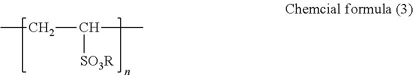
wherein R is H, Na, or K.

10. The target recognition molecule as set forth in claim 3,
wherein the electrostatically-charged segment is a segment having a polydextran sulfate building block represented by the following chemical formula (4) with n being not less than 1 nor more than 150. 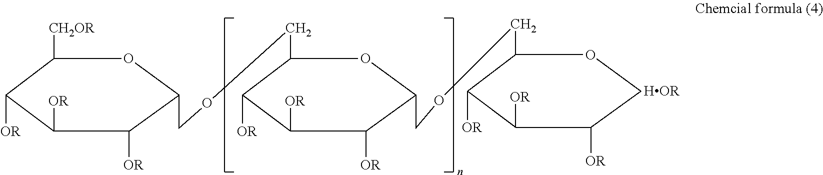
R═SO₃Na, SO₃H, or SO₃K

11. The target recognition molecule as set forth in claim 3,
wherein the electrostatically-charged segment is a segment having a polychondroitin sulfate building block represented by the following chemical formula (5) with n being not less than 1 nor more than 150. 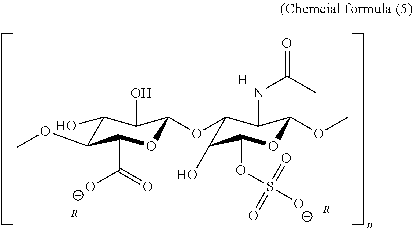
wherein R is H, Na, or K.

12. The target recognition molecule as set forth in claim 3,
wherein the electrostatically-charged segment is a segment having a polynucleotide building block represented by the following chemical formula (6) with n being not less than 3 nor more than 150. 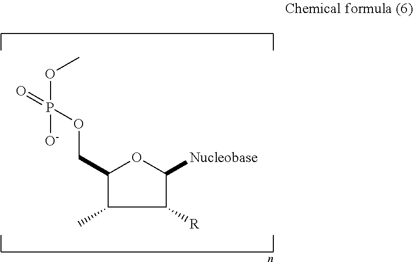
wherein R is H or OH.

13. The target recognition molecule as set forth in claim 3,
wherein the electrostatically-charged segment is a segment having a polyethylenimine building block represented by the following chemical formula (7). 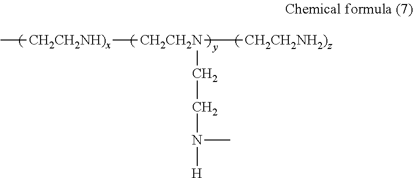
wherein x: y: z=0.5: 0.25: 0.25 and [x+y+z] is an integer not less than 3 nor more than 150.

14. The target recognition molecule as set forth in claim 3,
wherein the electrostatically-charged segment is a segment having a polyallylamine hydrochloride building block represented by the following chemical formula (8) with n being not less than 3 nor more than 150.

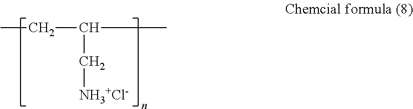

15. The target recognition molecule as set forth in claim 3,
wherein the electrostatically-charged segment is a segment having a polydiallyldimethylammonium chloride building block represented by the following chemical formula (9) with n being not less than 3 nor more than 150.

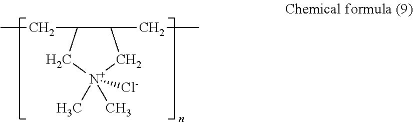

16. The target recognition molecule as set forth in claim 3,
wherein the electrostatically-charged segment is a segment having a polyvinylpyridine building block represented by the following chemical formula (10) with n being not less than 3 nor more than 150.

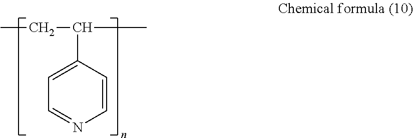

17. The target recognition molecule as set forth in claim 3,
wherein the electrostatically-charged segment is a segment having a peptide chain.

18. The target recognition molecule as set forth in claim 17,
wherein the peptide chain of the electrostatically-charged segment contains three or more acidic amino acid residues, one or more of which are selected from a group composed of an aspartic acid residue and a glutamic acid residue, and contains no basic amino acid residues such as an arginine residue and a lysine residue.

19. The target recognition molecule as set forth in claim 18,
wherein a content rate in the number of the acidic amino acid residues is 60% or more in the peptide chain of the electrostatically-charged segment.

20. The target recognition molecule as set forth in claim 18,
wherein the average isoelectric point of the peptide chain constituting the target recognition peptide segment is 8 or less, and wherein the average isoelectric point of the peptide chain constituting the electrostatically-charged segment is from 2.77 or more to 4.5 or less.

21. The target recognition molecule as set forth in claim 19,
wherein the average isoelectric point of the peptide chain constituting the target recognition peptide segment is 8 or less, and wherein the average isoelectric point of the peptide chain constituting the electrostatically-charged segment is from 2.77 or more to 4.5 or less.

22. The target recognition molecule as set forth in claim 3,
wherein
the electrostatically-charged segment contains no basic amino acid residues such as an arginine residue and a lysine residue, and contains 6 or more acidic amino acid residues, one or more of which are selected from a group composed of an aspartic acid residue and a glutamic acid residue;
a content rate in the number of the acidic amino acid residues is 60% or more in the peptide chain; and
two or less neutral amino acid residues are interposed between adjacent two of the acidic amino acid residues.

23. The target recognition molecule as set forth in claim 17,
wherein the peptide chain of the electrostatically-charged segment contains three or more basic amino acid residues, one or more of which are selected from a group composed of an arginine residue and a lysine residue, and contains no acidic amino acid residues such as an aspartic acid residue and a glutamic acid residue.

24. The target recognition molecule as set forth in claim 23,
wherein a content rate in the number of the basic amino acid residues is 60% or greater in the peptide chain of the electrostatically-charged segment.

25. The target recognition molecule as set forth in claim 23,
wherein the average isoelectric point of the peptide chain constituting the target recognition peptide segment is 6 or greater, and wherein the average isoelectric point of the peptide chain constituting the electrostatically-charged segment is from 8 or greater to 10.76 or less.

26. The target recognition molecule as set forth in claim 24,
wherein the average isoelectric point of the peptide chain constituting the target recognition peptide segment is 6 or greater, and wherein the average isoelectric point of the peptide chain constituting the electrostatically-charged segment is from 8 or greater to 10.76 or less.

27. The target recognition molecule as set forth in claim 3,
wherein
the electrostatically-charged segment contains no acidic amino acid residues such as an aspartic acid residue and a glutamic acid residue, and contains 6 or more basic amino acid residues, one or more of which are selected from a group composed of an arginine residue and a lysine residue;
a content rate in the number of the basic amino acid residues is 60% or greater in the peptide chain of the electrostatically-charged segment; and
two or less neutral amino acid residues are interposed between adjacent two of the basic amino acid residues.

28. The target recognition molecule as set forth in claim 3,
wherein the target recognition peptide segment contains a cysteine residue, and the electrostatically-charged segment is chemically linked to the sulfur atom in the cysteine residue.

29. The target recognition molecule as set forth in claim 3,
wherein either end of the target recognition peptide segment is a cysteine residue, and the electrostatically-charged segment is chemically linked to the sulfur atom in the cysteine residue.

30. The target recognition molecule as set forth in claim 3,
wherein the electrostatically-charged segment is chemically linked to the N-terminal or C-terminal of the amino acids constituting the target recognition peptide segment.

31. The target recognition molecule as set forth in claim 3,
wherein the target recognition peptide segment is a peptide that contains 3 or more and 19 or less amino acid residues.

32. The target recognition molecule as set forth in claim 3,
wherein the electrostatically-charged segment is further chemically linked to a base material immobilizing segment which is provided with a functional group for linkage to a base material.

33. A method for immobilizing a target recognition molecule onto a surface of an electrode formed in a microchannel of a microfluidic device, wherein the method comprises:
a target recognition molecule solution preparation step in which the target recognition molecule as set forth in claim 4 is dissolved in a solution to thereby adjust pH of the solution to pH that is equal to or more than an average isoelectric point of the target recognition peptide segment, and
a retaining step in which, with positive electric charges impressed to the electrode in the microchannel, the target recognition molecule containing solution is flowed in the microchannel so that the target recognition molecule in the solution is electrically absorbed and retained on the electrode surface.

34. A method for immobilizing a target recognition molecule onto a surface of an electrode formed in a microchannel of a microfluidic device, wherein the method comprises:
a target recognition molecule solution preparation step in which the target recognition molecule as set forth in claim 5 is dissolved in a solution to thereby adjust pH of the solution to pH that is equal to or less than an average isoelectric point of the target recognition peptide segment, and
a retaining step in which, with negative electric charges impressed to the electrode in the microchannel, the target recognition molecule containing solution is flowed in the microchannel so that the target recognition molecule in the solution is electrically absorbed and retained on the electrode surface.

35. A method for immobilizing a target recognition molecule onto a surface of an electrode formed in a microchannel of a microfluidic device, wherein the method comprises:
a target recognition molecule solution preparation step in which the target recognition molecule as set forth in claim 6 is dissolved in a solution to thereby adjust pH of the solution to more than 6.5 and less than 7.5, and
a retaining step in which, with either positive or negative electric charges impressed to the electrode in the microchannel, the target recognition molecule containing solution is flowed in the microchannel so that the target recognition molecule in the solution is electrically absorbed and retained on the electrode surface.

36. A method for immobilizing a target recognition molecule onto a surface of an electrode formed in a microchannel of a microfluidic device, wherein the method comprises:
a target recognition molecule containing solution preparation step in which the target recognition molecule as set forth in claim 20 is dissolved in a solution to thereby adjust pH of the solution to 6 or greater and 8.5 or less, and
a retaining step in which, with positive electric charges impressed to the electrode in the microchannel, the target recognition molecule containing solution is flowed in the microchannel so that the target recognition molecule in the solution is electrically absorbed and retained on the electrode surface.

37. A method for immobilizing a target recognition molecule onto a surface of an electrode formed in a microchannel of a microfluidic device, wherein the method comprises:
a target recognition molecule solution preparation step in which the target recognition molecule as set forth in claim 25 is dissolved in a solution to thereby adjust pH of the solution to 6 or greater and 8.5 or less, and
a retaining step in which, with negative electric charges impressed to the electrode in the microchannel, the target recognition molecule containing solution is flowed in the microchannel so that the target recognition molecule in the solution is electrically absorbed and retained on the electrode surface.

38. A method for immobilizing a target recognition molecule onto a surface of an electrode formed in a microchannel of a microfluidic device, wherein the method comprises:
a step for a target recognition molecule containing solution in which the target recognition molecule as set forth in claim 32 is dissolved in an aqueous solvent to thereby adjust the solution to a predetermined pH; and
a step in which, with electric charges having an opposite polarity to that of the electrostatically-charged segment in the target recognition molecule containing solution impressed to the electrode in the microchannel, the target recognition molecule containing solution is flowed in the microchannel so that the target recognition molecule in the solution is electrically absorbed and temporarily retained on the electrode surface, and then the target recognition molecule is immobilized on the electrode surface via a base material immobilizing segment of the target recognition molecule.

39. A target recognition molecular immobilization electrode plate comprising an electrode plate and the target recognition molecular as set forth in claim 3 which is immobilized on a surface of the electrode plate.

40. A particular molecule detection apparatus comprising a channel, an electrode plate disposed in the channel, and the target recognition molecule as set forth in claim 3 which is immobilized on a surface of the electrode plate.


SEQUENCE LISTING (14 sequences)

SEQ ID NO: 1 - protein, 11 aa, Homo sapiens
  MOD_RES (6)..(7): Phosphorylated-Ser
GRPRTSSFAEG

SEQ ID NO: 2 - protein, 8 aa, Artificial Sequence
  Description of Artificial Sequence Synthetic peptide
DDDDDDDD

SEQ ID NO: 3 - protein, 19 aa, Artificial Sequence
  Description of Artificial Sequence Synthetic peptide
GRPRTSSFAEGDDDDDDDD

SEQ ID NO: 4 - protein, 20 aa, Artificial Sequence
  Description of Artificial Sequence Synthetic peptide
GRPRTSSFAEGDDDDDDDDC

SEQ ID NO: 5 - protein, 20 aa, Artificial Sequence
  Description of Artificial Sequence Synthetic peptide
GRPRTSSFAEGDDDDDDDDC

SEQ ID NO: 6 - protein, 7 aa, Homo sapiens
  MOD_RES (5)..(5): Phosphorylated-Ser
LRRASLG

SEQ ID NO: 7 - protein, 10 aa, Artificial Sequence
  Description of Artificial Sequence Synthetic peptide
RRRRRRRRRR

SEQ ID NO: 8 - protein, 17 aa, Artificial Sequence
  Description of Artificial Sequence Synthetic peptide
LRRASLGRRRRRRRRRR

SEQ ID NO: 9 - protein, 21 aa, Artificial Sequence
  Description of Artificial Sequence Synthetic peptide
LRRASLGRRRAHKKKTRKRPK

SEQ ID NO: 10 - protein, 18 aa, Artificial Sequence
  Description of Artificial Sequence Synthetic peptide
LRRASLGRRRRRRRRRRC

SEQ ID NO: 11 - protein, 18 aa, Artificial Sequence
  Description of Artificial Sequence Synthetic peptide
LRRASLGRRRRRRRRRRC

SEQ ID NO: 12 - protein, 8 aa, Artificial Sequence
  Description of Artificial Sequence Synthetic peptide
LRRASLGK

SEQ ID NO: 13 - protein, 8 aa, Artificial Sequence
  Description of Artificial Sequence Synthetic peptide
LRRASLGC

SEQ ID NO: 14 - protein, 9 aa, Artificial Sequence
  Description of Artificial Sequence Synthetic peptide
DDDDDDDDC